(19) United States
(12) Patent Application Publication
(10) Pub. No.: US 2023/0086394 A1
(43) Pub. Date: Mar. 23, 2023

(54) ELECTRODE ASSEMBLY

(71) Applicant: GYRUS MEDICAL LIMITED, St Mellons (GB)

(72) Inventors: Thomas LLOYD, St Mellons (GB); James DICKSON, St Mellons (GB); Nicholas TULP, St Mellons (GB); Sunny RUPALIA, St Mellons (GB)

(73) Assignee: GYRUS MEDICAL LIMITED, St Mellons (GB)

(21) Appl. No.: 17/945,648

(22) Filed: Sep. 15, 2022

Related U.S. Application Data
(60) Provisional application No. 63/245,962, filed on Sep. 20, 2021.

(30) Foreign Application Priority Data
Sep. 21, 2021 (GB) 2113433.3

(51) Int. Cl. A61B 18/14

(52) U.S. Cl. CPC A61B 18/148; A61B 2018/00071

(57) ABSTRACT

The present disclosure provides an electrode assembly wherein an electrically conductive material is configured to extend into an insulating moulding via an opening in a wall of said moulding to electrically contact an active tip placed into the insulating moulding and thereby retain the active electrode therein, such that the height and space required to anchor the active electrode within the moulding is greatly reduced while still providing the necessary retention strength and electrical connection. As such, the electrode assembly retains and connects the active tip with a conductive element that is mounted within the insulating moulding.

### TECHNICAL FIELD

The present invention relates to an electrode assembly. More specifically, the present invention relates to a method of manufacturing an electrode assembly for use in an electrosurgical instrument.

### BACKGROUND TO THE INVENTION AND PRIOR ART

Surgical instruments, including radio frequency (RF) electrosurgical instruments, have become widely used in surgical procedures where access to the surgical site is restricted to a narrow passage, for example, in minimally invasive “keyhole” surgeries.

Electrosurgical instruments provide advantages over traditional surgical instruments in that they can be used for coagulation and tissue sealing purposes (single-purpose), or incorporate both coagulation/ablation and mechanical shaving functionality (dual-purpose).

Electrosurgical instruments are commonly comprised of an active electrode encased within an insulator, for example, a ceramic body, wherein at least a portion of the active electrode is exposed for treating tissue, such as the instrument as described in U.S. Pat. No. 8,932,285.

The active electrode tip (or ‘active tip’) of the electrosurgical instrument needs to be securely retained by the insulator by some way. Single-purpose electrosurgical devices have leveraged the internal space within the electrode to house a robust electromechanical connection between the active tip and the rest of the device. The electromechanical connection may achieved be welding, crimping or riveting of at least two mating parts. This arrangement ensures that the active tip is securely retained in the insulator with sufficient mechanical strength to prevent the active tip becoming separated from the insulator. This arrangement also ensures electrical conductivity of the active tip is maintained during use.

A typical RF electrosurgical instrument is shown in FIG. 2, with a typical active electrode retention method. In the generic instrument shown in FIG. 2, the active electrode 14 is housed inside the electrode assembly 12 and occupies the internal space 22. The active electrode 14 is secured to the electrode assembly 12 by mechanically engaging the electrode to the outer casing 20.

However, in dual-purpose electrosurgical devices, the internal space, which typically anchors the active tip, is occupied by an inner shaver blade. The area available for retaining and anchoring the active tip is significantly smaller in dual-purpose electrosurgical devices compared to single-purpose devices. Consequently, it is more difficult to anchor the active tip in a dual-purpose electrode while still maintaining mechanical strength and electrical conductivity. If the active tip is not securely retained, the active tip may become loose and fall out of the insulating portion or loose electrical conductivity and render the electrosurgical device unusable or unsafe. This can be particularly problematic if this occurs during surgery, especially in the context of keyhole procedures.

### SUMMARY OF THE INVENTION

The present disclosure addresses the above problem, by providing an electrode assembly wherein an electrically conductive material is configured to extend into an insulating moulding via an opening in a wall of said moulding to electrically contact an active tip placed into the insulating moulding and thereby retain the active electrode therein, such that the height and space required to anchor the active electrode within the moulding is greatly reduced while still providing the necessary retention strength and electrical connection. As such, the electrode assembly retains and connects the active tip with a conductive element that is mounted within the insulating moulding.

In view of the above, from a first aspect, the present disclosure relates to an electrode assembly for use in an end effector of an electrosurgical instrument, the electrode assembly comprising: a first electrode; and an outer moulding, wherein the outer moulding comprises a cavity configured to receive the first electrode; and an electrically conductive material, wherein the electrically conductive material is configured to extend into the outer moulding such that it electrically contacts the first electrode, the electrically conductive material being coupled to the first electrode to thereby retain the first electrode within the outer moulding.

As such, the electrically conductive material anchors the first electrode to the outer moulding, whilst at the same time providing an electrical connection for delivering power to the first electrode. By passing the conductive material through the outer moulding and then securing it to the first electrode, the height required to anchor the first electrode is minimized whilst maintaining a strong mechanical connection. This arrangement also helps prevent movement of the first electrode within the cavity, and ensures that the first electrode, outer moulding and electrically conductive material cannot be separated without permanent deformation or fracture.

In some embodiments, the electrically conductive material comprises a first extending arm (for example, a pin-shaped conductor). In other embodiments, the electrically conductive material comprises a first extending arm and a second extending arm (for example, an L-shaped conductor).

In some embodiments, the electrically conductive material comprises a U-shaped conductor with at least a first extending arm and a second extending arm. By having a generally U-shaped conductor with arms, an improved cross-sectional area for the current is provided when compared to using a single length of wire. That is to say, the U-shaped conductor provides a greater cross-sectional area for the current to pass, which reduces the current density in the first extending arm and the second extending arm. The U-shaped conductor therefore permits a smaller current density by providing a greater area, and also reduces any unwanted joule heating effects. This shape is also advantageous as it is easy to manufacture and allows for a simple assembly of the electrode assembly.

In some embodiments, the electrically conductive material extends into the first electrode. In this respect, the electrically conductive material may engage with an aperture of the first electrode to thereby prevent withdrawal. That is to say, the distal end of the electrically conductive material is received by the first electrode. In the example where the electrically conductive material comprises a U-shaped conductor, the distal end of the first extending arm and the second extending arm may be inserted through a proximal wall of outer moulding and the received within two holes disposed within the proximal edge of the first electrode. By inserting an electrically conductive material with a high flexural modulus into the first electrode via the outer moulding, movement of the first electrode within the outer moulding is minimised.

In some embodiments, the conductive material extends into the cavity at a first proximal end, the conductive material being secured to the outer moulding at a second distal end. By securing the conductive material at the second distal end, the mechanical strength of the arrangement is improved by providing additional structural support to the electrode assembly.

In some embodiments, the first electrode is inserted into the cavity such that it abuts the conductive material therethrough. For example, in the case where the conductive material is secured at both ends of the outer moulding such that it passes through the cavity, the first electrode may be then inserted to the cavity such that it sits onto the conductive material. The first electrode may then be bonded to the conductive material. In doing so, the first electrode is mechanically coupled to the conductive material to thereby prevent lateral abduction of the first electrode.

In some embodiments, the electrically conductive material is configured to elastically deflect upon insertion to the outer moulding to thereby contact the first electrode. As the electrically conductive material deflects, it creates a contact force which ensures electrical continuity between the electrode and the conductive material, whilst at the same time creating a mechanical connection. In this respect, the electrically conductive material may elastically deflect to engage an aperture of the first electrode.

In some embodiments, the electrode assembly further includes a connector in contact with the electrically conductive material, wherein the electrically conductive material is configured to displace the connector from a first position to a second position, such that the connector electrically contacts the first electrode in the second position.

In some embodiments, the electrically conductive material is configured to translate in a longitudinal direction towards the distal end of the electrode assembly. In this respect, a translatable connector may be provided that is connected to the electrically conductive material so as to translate the electrically conductive material in the longitudinal direction, the translatable connector being arranged on the outside of the outer moulding to allow translation by the user.

In some embodiments, the connector has a first end and a second end, and at least one of the first and second ends is not in the same plane as the electrically conductive material, such that displacement of the electrically conductive material displaces one of the first and second ends into the same plane as the electrically conductive material.

In some embodiments, the connector and the electrically conductive material partially extend into the outer moulding in the second position. The connector is thus displaced so that it sits in an opening in the outer moulding. The connector and electrically conductive material are therefore in electrical connection with each other. This arrangement provides a one-way mechanical displacement of the connector as the connector cannot be returned to the first position once it has been displaced to the opening in the outer moulding. The connector thus provides a one-way mechanical feature that prevents disassembly of the electrode assembly.

In some embodiments, the electrically conductive material is metallically bonded to the first electrode by at least one of welding, crimping, riveting, brazing or another joining process. This provides electrical conductivity and continuity between the first electrode and the conductive material. Metallically bonding also provides a stiffness that helps to provide a strong mechanical connection between the electrode and the conductive material.

In some embodiments, the first electrode is an active electrode. In this respect, the first electrode may comprise a tissue treatment portion, wherein the tissue treatment portion is provided by a surface of the first electrode.

In some embodiments, the electrically conductive material is a wire. Preferably, the wire has a high flexural modulus. In some embodiments, the electrically conductive material comprises copper, stainless steel, or tungsten alloy. Tungsten in particular has sufficient flexural stiffness to resist deflection under load and conductivity without an unacceptable level of heating in the electrode assembly.

In some embodiments, the electrode assembly further includes a lumen that extends along the length of the electrode assembly, wherein the electrically conductive material is arranged parallel with the lumen. The lumen will contain other features of the electrosurgical instrument, such as a suction lumen for removing and delivering fluid to the tissue treatment site, and the mechanical shaver. As such, the conductive material is arranged alongside the lumen, leaving space within the lumen for the other features of instrument.

In some embodiments, the electrode assembly further includes a mechanical shaver portion.

Preferably, the outer moulding comprises an insulating material, for example, a ceramic material.

A further aspect provides an electrosurgical instrument comprising: an instrument shaft having a longitudinal axis; and an electrode assembly at one end of the shaft, the electrode assembly comprising a first electrode; an outer moulding, wherein the outer moulding comprises a cavity configured to receive the first electrode; and an electrically conductive material; wherein the electrically conductive material is configured to extend through the outer moulding such that it electrically contacts the first electrode, the electrically conductive material being coupled to the first electrode to thereby retain the first electrode within the outer moulding.

Another aspect provides a method of manufacturing an electrode assembly for use in an end effector of an electrosurgical instrument, comprising providing a first electrode; providing an outer moulding, wherein the outer moulding comprises a cavity configured to receive the first electrode; inserting an electrically conductive material to the outer moulding such that the electrically conductive material contacts the first electrode, and coupling the electrically conductive material to the first electrode to thereby retain the first electrode within the outer moulding.

In some embodiments, the method may further comprise inserting the electrically conductive material into the cavity at a first proximal end such that it extends therethrough, and securing the electrically conductive material to the outer moulding at a second distal end.

In some embodiments, the method may further comprise inserting the electrically conductive material into the first electrode. For example, the electrically conductive material may be inserted to one or more holes in a proximal edge of the first electrode.

In some embodiments, coupling the electrically conductive material to the first electrode comprises metallically bonding the electrically conductive material to the first electrode. For example, the electrically conductive material is metallically bonded to the first electrode by at least one of welding, crimping, riveting, brazing or another joining process.

An electrosurgical system may also be provided, comprising an RF electrosurgical generator, and an electrosurgical instrument comprising an electrode assembly, the electrode assembly comprising a first electrode, an outer moulding, wherein the outer moulding comprises a cavity configured to receive the first electrode; and an electrically conductive material, wherein the electrically conductive material is configured to extend into the outer moulding such that it electrically contacts the first electrode, the electrically conductive material being coupled to the first electrode to thereby retain the first electrode within the outer moulding.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 is a schematic diagram of an electrosurgical system including an electrosurgical instrument according to an embodiment of the present invention.

FIG. 2 is a cross-sectional diagram of an electrode according to the prior art;

FIG. 3 is a cross-section side view of an electrode assembly manufactured according to the present invention;

FIG. 4 is a top view of an electrode assembly according to the present invention;

FIG. 5 is an isometric view of the outer casing of the electrode assembly according to the present invention;

FIG. 6 is a top view of the outer casing of the electrode assembly according to the present invention;

FIG. 7 is a perspective view of an active electrode of the electrode assembly according to the present invention;

FIG. 8 is a top view of an active electrode of the electrode assembly manufactured according to the present invention;

FIG. 9 is a cross-sectional side view of an electrode assembly manufactured according to the present invention;

FIG. 10 is a top view of an electrode assembly according to the present invention;

FIG. 11 is a cross-sectional side view of an electrode assembly according to the present invention;

FIG. 12 is a view of the electrode assembly according to the present invention.

### DESCRIPTION OF THE EMBODIMENTS

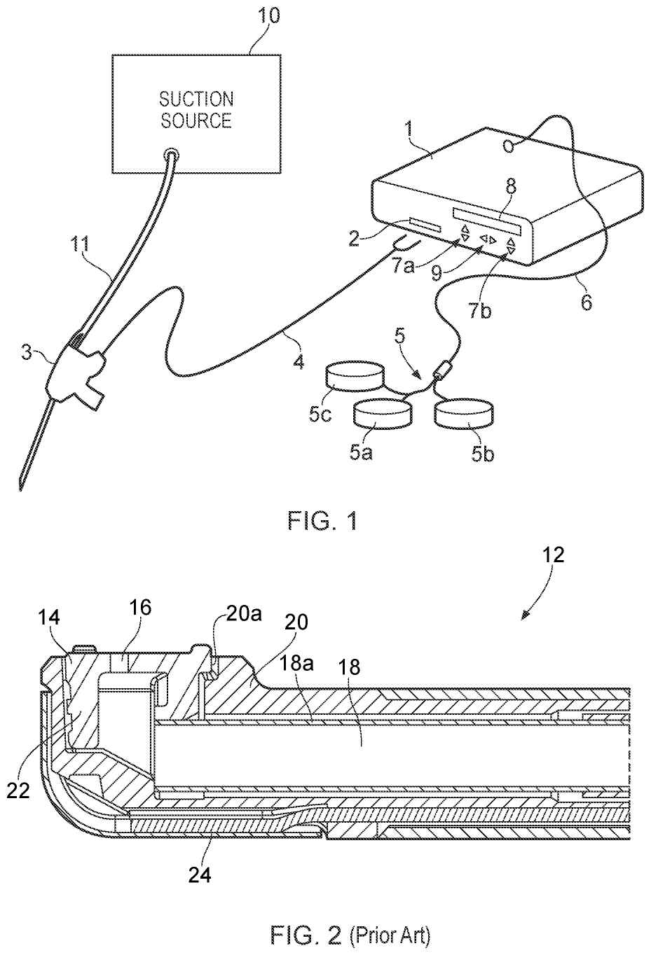

FIG. 1 shows an electrosurgical apparatus including an electrosurgical generator 1 having an output socket 2 providing a radio frequency (RF) output, via a connection cord 4, for an electrosurgical instrument 3 having an end effector configured to provide a mechanical shaving function, as well as electrosurgical cutting and coagulation functions. The instrument 3 may also comprise irrigation and suction tubes 11 which are connected to an irrigation fluid and suction source 10. Activation of the generator 1 may be performed from the instrument 3 via a handswitch (not shown) on the instrument 3, or by means of a footswitch unit 5 connected separately to the rear of the generator 1 wirelessly or by a footswitch connection cord 6. In the illustrated embodiment, the footswitch unit 5 has three footswitches 5a, 5b and 5c for selecting a mechanical shaving mode, a coagulation mode or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

FIG. 2 shows a single-purpose electrode assembly 12 with an active electrode 14, as currently known in the art. The electrode assembly 12 is designed for ablation of tissue. The active tip has a suction hole 16, which may be the opening to a lumen 18 within the insulating outer casing 20 for use in delivering fluids to and from the active electrode 14. The lumen 18 may be enclosed by an active tubular portion 18a extending from the active electrode 14 for providing a robust electromechanical connection between the active electrode 14 and the rest of the device. The active electrode 14 is housed inside the electrode assembly occupies an internal space 22. The active electrode 14 is secured to the electrode assembly 12 by mechanically engaging the electrode with the outer casing 20. The electrode assembly 12 shown in FIG. 2 may also have a return electrode 24 connected to the insulated outer casing 20.

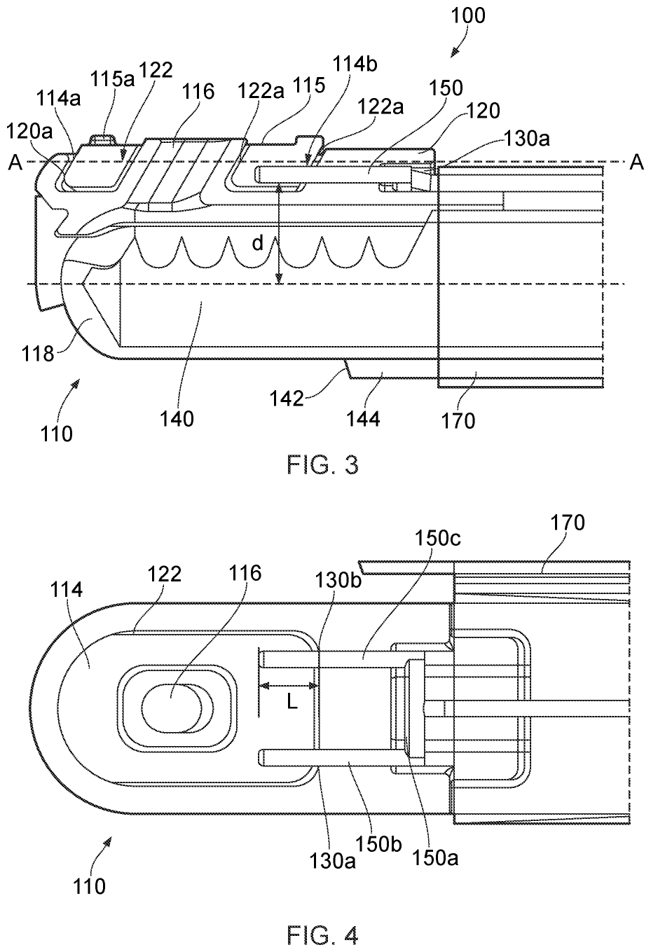

FIGS. 3 and 4 show an electrode assembly 100 according to the present invention. In contrast to the electrode assembly described in FIG. 2, electrode assembly 100 is incorporated into an instrument 110 which is capable of both coagulating tissue and ablating tissue using the electrode assembly 100, as well as implementing a shaver blade 140 to mechanically cut the tissue through a cutting window 142 in the shaver casing 144. The shaver blade 140 occupies the majority of the lumen 118 and reduces the amount of internal space available for the electrode assembly 100 to be secured to the outer moulding 120 of instrument 110. During use, the shaver blade 140 cuts tissue that is presented adjacent to the cutting window 142, that is, the tissue site is located in a direction orthogonal longitudinal axis of the instrument. Similarly, the electrode assembly 100 coagulates or ablates tissue that is presented adjacent to the active electrode 114. The instrument 110 may be used to cut tissue at a tissue site before being rotated to coagulate or ablate the tissue and suction the removed tissue away, permitting very quick tissue removal. Alternatively, the instrument 110 may be used for only cutting tissue or ablating/coagulating the tissue site as needed.

The outer moulding 120 has a cavity 122 denoted by the walls 122a, and two openings, 130a and 130b in the proximal wall. Only one opening, 130a is shown in FIG. 3. The cavity 122 is configured to receive the active electrode 114. It will be appreciated that any suitable cavity 122 may be provided according to the desired final configuration of the electrode 114. The outer moulding 120 is made of any suitable material, for example, a ceramic material such as alumina, zirconia toughened alumina (ZTA), yttria stabilized zirconia (YTZP) or the like. The active electrode 114 also has an external tissue treatment portion 115, which protrudes from the outer moulding 120. This exposed treatment portion 115 may have ridges for use in ablation and vaporisation of tissue, or protrusions 115a which can be used to contact tissue sites. The outer moulding 120 has a suction hole 116 that is integrally formed within the moulding. The suction hole 116 is in fluid communication with the suction lumen 118 of the instrument 110. During use, fluid, tissue fragments, bubbles or other debris in the vicinity of the electrode assembly 100 can be aspirated from the surgical site. As noted above the tissue to be treated is adjacent to either the electrode assembly 100, or adjacent to the cutting window 142, in a direction orthogonal to the long axis of the instrument. The active electrode 114 is shaped to accommodate a suction hole 116, as shown in FIG. 4. Alternatively, the active electrode 114 may have a suction hole integrated in the external tissue treatment portion 115.

The outer moulding openings 130a and 130b are configured to receive an active wire arrangement 150 connected to a power supply (not shown) The active wire arrangement is generally U-shaped as shown in FIG. 4, with two extending arms 150b and 150c and an interconnecting arm 150a. The active wires 150 may be any material suitable for connecting to the active electrode 114, for example, a metal, such as copper, tungsten or steel. Preferably, the active wires 150 are made from Tungsten, as it has high flexural stiffness and can be conductive without an unacceptable amount of heating. The active wire arrangement 150 is inserted to two holes in the proximal edge of the active electrode 114, to thereby provide an electrical contact. The active wire arrangement 150 can be mechanically joined to the active tip 114, for example, by laser welding or metallic bonding. Alternatively, the wire arrangement 150 may be coupled to the active electrode 114 via some other one-way mechanical feature such as a snap-fit arrangement. By coupling the wire arrangement 150 and the active electrode 114 in this way, proximal abduction of the wire arrangement 150 is prevented. The active wire arrangement 150 also has high flexural stiffness to resist load during assembly of the electrode assembly 100 and during use of the instrument 110. By passing the active wire arrangement 150 through the openings 130a and 130b in the outer moulding 120 and into the active electrode 114, the active electrode 114, active wire arrangement 150 and outer moulding 120 cannot be separated without deforming or fracturing the whole assembly.

The outer moulding openings 130a and 130b are preferably formed so that the active wire arrangement runs parallel to the lumen 118 of the instrument, whilst occupying as little space as possible. While FIG. 3 shows the active wire arrangement 150 to be a distance d from the centre of the lumen 118, it will be understood that the active wire arrangement 150 may be closer to the external treatment portion 115 or closer to the lumen 118. This arrangement also ensures that the electrode assembly 100 is mechanically secure, with reliable electrical connection between the active wire arrangement 150 and the active electrode 114 while occupying as little height as possible within the instrument 110.

FIG. 4 shows a cross-sectional top view of the electrode assembly 100 shown in FIG. 3. As shown, the conductive wire arrangement 150 passes through the openings 130a and 130b of the outer moulding, with the arms 150b and 150c extending a distance L into the active electrode 114. The distance L may be any suitable distance to ensure the arms 150b and 150c can be securely connected to the active electrode 114 through any suitable means, such as metallic bonding. As shown in FIG. 3, the conductive wire arrangement 150 is arranged to be inserted into the active electrode 114. That is to say, the conductive wire arrangement 150 is distanced from the surface defining the external tissue portion 115. By arranging the conductive wire arrangement 150 inside the active electrode 114, this helps anchor the active electrode 114 and prevent separation of the active electrode 114, wire arrangement 150 and outer moulding 120.

To assemble the electrode arrangement 100, an outer moulding 120 is provided, the moulding 120 being formed of some suitable insulating material, such as a ceramic. The active electrode 114 is then placed into the cavity 122 of the ceramic moulding 120. In this example, the distal edge 114a of the active electrode 114 abuts the inner edge 120a of the cavity 122. The conductive wire arrangement 150 is inserted through the openings 130a and 130b and into the proximal edge 114b of the active electrode 114. The wire arrangement 150 is then coupled to the active electrode 114, preferably by metallic bonding, or some other suitable method. The conductive wire arrangement 150 may be connected to the active electrode 114 by, for example, laser welding, soldering, brazing or any other suitable method of forming a metallic bond. Alternatively, if the active electrode 114 is not metallically bonded to the conductive wire arrangement 150, it may be formed so that it deflects elastically upon insertion through openings 130a and 130b. This creates a contact force that ensures electrical continuity between the conductive wire arrangement 150 and the active electrode 114. The conductive wire arrangement 150 may also be bonded to the ceramic moulding 120. Due to the flexural stiffness of the conductive wire arrangement 150, the conductive wire arrangement helps prevent movement of the active electrode 114 within the cavity 122. It will however be appreciated that the conductive wire arrangement 150 may be secured to the outer moulding 120 prior to assembling the electrode assembly 100.

While the conductive wire arrangement 150 is described as being generally U-shaped, it will be understood that any suitable configuration may be used, for example, a single wire, pin-shaped, L-shaped, I-shaped, a trident shape or any other suitable shape. Similarly, while the openings 130a and 130b are shown to be two separate openings along the side of the outer moulding 120, it will be appreciated that there may only be one opening, or the openings could be located at any suitable location in the outer moulding.

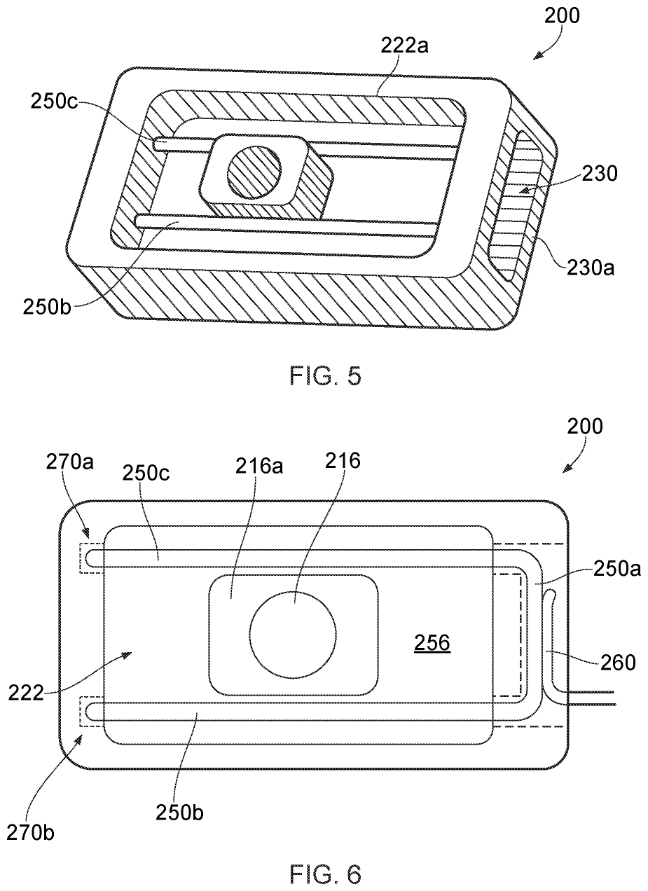

FIGS. 5-9 show the components of another example of the electrode assembly 200 with the rest of instrument 110 omitted for clarity. FIGS. 5 and 6 show the outer moulding 220. The outer moulding 220 has an internal cavity 222 which is denoted by the walls 222a, and an opening 230, denoted by boundaries 230a. The electrode assembly 200 has a suction support 216a which defines a suction hole 216. The suction hole 216 will be arranged in fluid communication with the lumen 118 of the instrument 110 (not shown). In this example, the conductive wire arrangement 250 is generally U shaped with two extending arms 250b and 250c and an interconnecting arm 250a. The two extending arms 250b and 250c of the conductive wire arrangement 250 run parallel with the length of the casing. The two extending arms 250b and 250c are secured to the distal end of the outer moulding 220 at a first position 270a and a second position 270b. The interconnecting arm 250a of the conductive wire arrangement 250 is in electrical contact with a further conductive wire 260, for example, via welding, soldering, brazing or crimping, the further conductive wire 260 leading back to an electrical source. While the conductive wire arrangement 250 is shown to be one continuous wire, it will be understood that number of connecting wires may be used, or two separate wires.

As shown in FIGS. 5 and 6, the suction support 216a is secured in between the receiving cavity 256 of the arms 250b and 250c. This provides structural support to both the conductive wire arrangement 250 and the suction support 216a. Providing a generally U-shaped wire arrangement 250 with arms 250b and 250c allows for a greater cross-sectional area for the current to pass, which reduces the current density in the arms 225b and 225c when compared to using a single length of wire. Furthermore, this shape is also easy to manufacture and allows for a simple assembly of the electrode assembly 200. In this example, the conductive wire arrangement 250 is inserted into the outer moulding 220 through the opening 230.

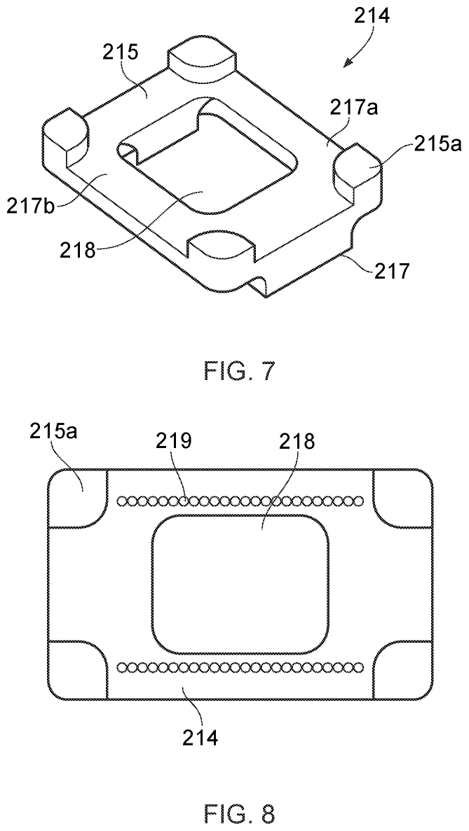

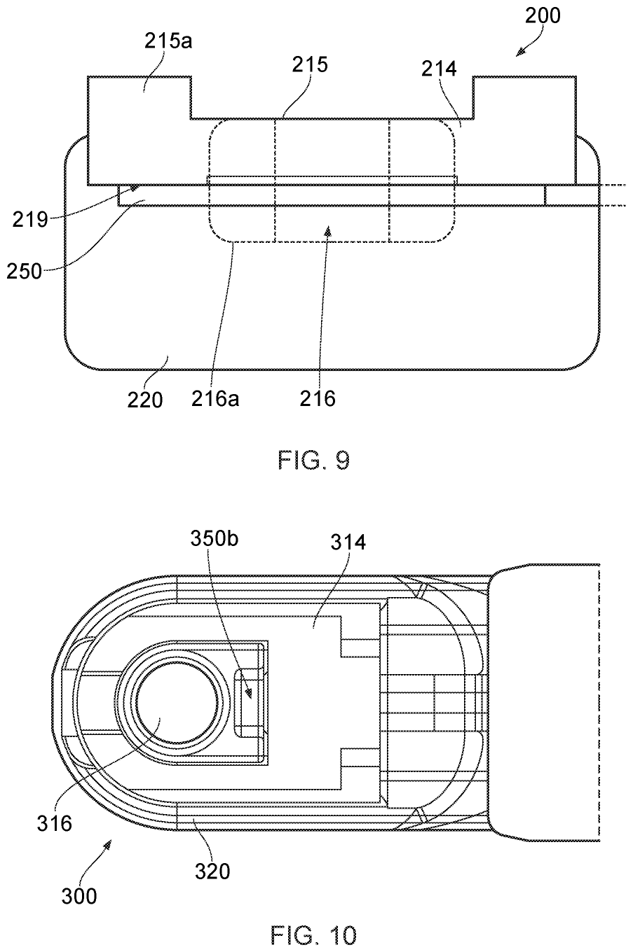

Once the conductive wire arrangement 250 and the suction support 216a are secured in the outer moulding 220, the active electrode 214 is introduced which is shown in greater detail in FIGS. 7 to 9. The active electrode 214 has a body portion 217 with two flanges, 217a and 217b. The active electrode 214 also has an exposed treatment portion 215 which may have ridges for use in ablation and vaporisation of tissue, or protrusions 215a which can be used to contact tissue sites. The active electrode 214 also has an opening 218 to allow fluid to pass through the body of the active electrode 214 via the suction support 216a. As shown in FIG. 9, the active electrode 214 is inserted to the cavity (not shown) of the outer moulding 220, such that it contacts the conductive wire arrangement 250 and the recess 218 is aligned with the suction support 216a. That is to say, the active electrode 214 is arranged to abut at least one of the arms 250a and 250b of the conductive wire arrangement 250. The active electrode 214 is secured to the conductive wire arrangement 250 by welding the active electrode 214 to the arms 250b and 250c, indicated by exemplary contact lines 219. As the conductive wire arrangement 250 is secured to the outer moulding 220 and electrically connected to the active electrode 214, it provides a secure arrangement that ensures that the active electrode and/or the conductive wire arrangement cannot be separated from the outer moulding 220 without fracturing or breaking the assembly.

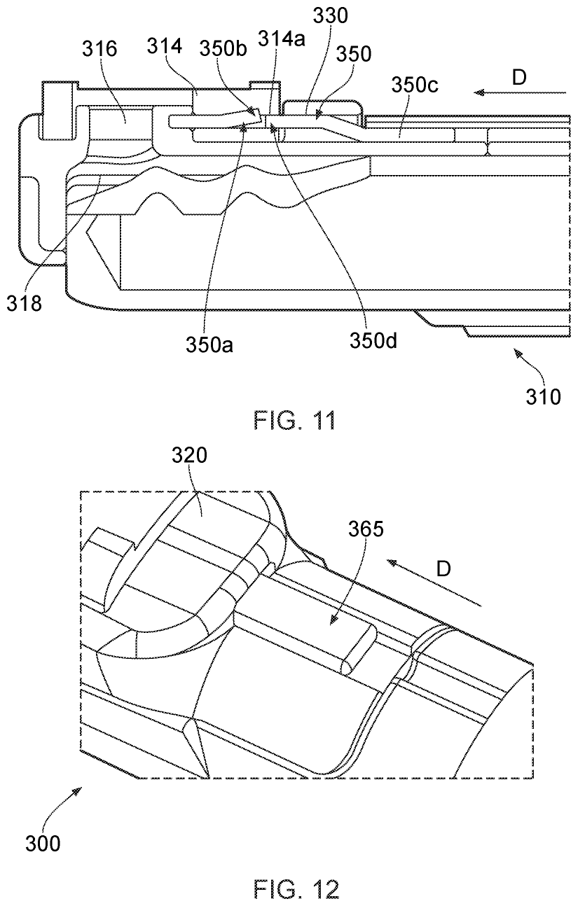

FIG. 10-12 show a further example of the present invention. In this arrangement, the electrode assembly 300 has an active electrode 314 which is retained in an outer moulding 320, similar to the aforementioned examples. As with the electrode assembly 100 shown in FIGS. 3 and 4, the electrode assembly 300 comprises a suction hole 316 which is again in fluid communication with the lumen 318 of the instrument 310. In this example, the conductive wire arrangement 350 extends through a channel 330 in the outer moulding 320 into a lumen 314a. A translatable connector 365, as shown in FIG. 12, is provided on the outside of the electrode assembly 300 that is then connected to the active wire arrangement 350. In the first position, the conductive wire arrangement 350 comprises a spade-style connector 350a which is disposed upwards from the upper face of a conductive wire 350c when at rest, as shown in FIG. 11. That is to say, the end face 350b of the spade style connector 350a is not aligned with the end face 350d of the conductive wire 350c. The end face 350b of the spade style connector 350a is thus not in the same plane as the conductive wire 350c.

In the second position, the active wire 350c is translated into the lumen 314a when the connector 365 is translated in a direction D, that is in a longitudinal direction away from the suction hole 316 towards the distal end of the electrode assembly 300. This causes the spade-style connector 350a to be forced downwards so that it presses up against the inside of the lumen 314a to form an electrical contact with the active electrode 314. The end face 350b of the spade style connector 350a is therefore aligned with the end face 350d of the conductive wire 350c. Once the spade-style connector 350a is in the second position, it is not possible to return to the first position by translating the connector 365 in the opposite direction to D as the spade-style connector 350a is located in the lumen 314a. In the second position, the active electrode 314 is connected to the energy supply to perform the desired function. In the second position, both the spade-style connector 350a and the conductive wire 350c are partially located in the lumen 314a. Therefore, the spade-style connector 350a acts as a one-way mechanical feature to activate the active electrode 314. This arrangement also helps prevent disassembly of the electrode assembly. In this respect, the active wire arrangement 350 comprises a snap-fit feature such that it engages with the lumen 314a and cannot be withdrawn away from the active electrode 314. The active wire arrangement 350 will then be connected to some other wired means back to an energy supply for delivering RF power to the active electrode 314. As mentioned previously, the active electrode 314 may also be welded to the distal end 350b of the active wire arrangement 350 to ensure a strong mechanical and electrical connection.

Where the word ‘or’ appears this is to be construed to mean ‘and/or’ such that items referred to are not necessarily mutually exclusive and may be used in any appropriate combination.

Although the invention has been described above with reference to one or more preferred embodiments, it will be appreciated that various changes or modifications may be made without departing from the scope of the invention as defined in the appended claims.

1. An electrode assembly for use in an end effector of an electrosurgical instrument, the electrode assembly comprising:
a first electrode; and
an outer moulding, wherein the outer moulding comprises a cavity configured to receive the first electrode; and
an electrically conductive material;
wherein the electrically conductive material is configured to extend into the outer moulding such that it electrically contacts the first electrode, the electrically conductive material being coupled to the first electrode to thereby retain the first electrode within the outer moulding, wherein the electrically conductive material comprises a first extending arm and a second extending arm.

2. An electrode assembly according to claim 1, wherein the electrically conductive material comprises a U-shaped conductor with at least the first extending arm and the second extending arm.

3. An electrode assembly according to claim 1, wherein the electrically conductive material extends into the first electrode.

4. An electrode assembly according to claim 1, wherein the conductive material extends into the cavity at a first proximal end, the electrically conductive material being secured to the outer moulding at a second distal end.

5. An electrode assembly according to claim 1, wherein the first electrode is inserted into the cavity such that it abuts the electrically conductive material therethrough.

6. An electrode assembly according to claim 1, wherein the first electrode is bonded to the electrically conductive material.

7. An electrode according to claim 1, wherein the electrically conductive material is configured to elastically deflect upon insertion to the outer moulding to thereby contact the first electrode.

8. An electrode assembly according to claim 7, wherein the electrically conductive material elastically deflects to engage with an aperture of the first electrode.

9. An electrode assembly according to claim 1, wherein the electrically conductive material is metallically bonded to the first electrode by at least one of welding, crimping, riveting, brazing or another joining process.

10. An electrode assembly according to claim 1, wherein the first electrode is an active electrode.

11. An electrode assembly according to claim 1, wherein the electrically conductive material is a wire.

12. An electrode assembly according to claim 1, wherein the electrically conductive material comprises copper, steel, or tungsten alloy.

13. An electrode assembly according to claim 1, further comprising a lumen that extends along the length of the electrode assembly, wherein the electrically conductive material is arranged parallel with the lumen.

14. An electrode assembly according to claim 1, wherein the electrode assembly further comprises a mechanical shaver portion.

15. An electrode assembly according to claim 1, wherein the outer moulding comprises an insulating material.

16. An electrosurgical instrument comprising:
an instrument shaft having a longitudinal axis; and
an electrode assembly at one end of the shaft, the electrode assembly comprising a first electrode; and
an outer moulding, wherein the outer moulding comprises a cavity configured to receive the first electrode; and
an electrically conductive material;
wherein the electrically conductive material comprises a first extending arm and a second extending arm and;
wherein the electrically conductive material is configured to extend through the outer moulding such that it electrically contacts the first electrode, the electrically conductive material being coupled to the first electrode to thereby retain the first electrode within the outer moulding.

17. A method of manufacturing an electrode assembly for use in an end effector of an electrosurgical instrument, comprising:
providing a first electrode;
providing an outer moulding, wherein the outer moulding comprises a cavity configured to receive the first electrode;
providing an electrically conductive material comprising a first extending arm and a second extending arm;
inserting the electrically conductive material to the outer moulding such that the electrically conductive material contacts the first electrode; and
coupling the electrically conductive material to the first electrode to thereby retain the first electrode within the outer moulding

18. A method of manufacturing the electrode assembly according to claim 17, further comprising:
inserting the electrically conductive material into the cavity at a first proximal end such that it extends therethrough; and
securing the electrically conductive material to the outer moulding at a second distal end.

19. A method of manufacturing the electrode assembly according to claim 18, further comprising inserting the electrically conductive material into the first electrode.

20. A method of manufacturing the electrode assembly according to claim 19, wherein coupling the electrically conductive material to the first electrode comprises metallically bonding the electrically conductive material to the first electrode.
